(19) United States
(12) Patent Application Publication
(10) Pub. No.: US 2008/0033261 A1
(43) Pub. Date: Feb. 7, 2008

(54) Measuring Blood Glucose Concentration

(76) Inventor: Philipp N. Zeller, Stallikon (CH)

Correspondence Address: FISH & RICHARDSON, PC, P.O. BOX 1022, MINNEAPOLIS, MN, 55440-1022, US

(21) Appl. No.: 10/558,568

(22) PCT Filed: May 28, 2004
(86) PCT No.: PCT/IB04/02013

§ 371 Date: Feb. 13, 2007

(30) Foreign Application Priority Data
May 28, 2003 (GB) 0312151.4

(51) Int. Cl. A61B 5/00

(52) U.S. Cl. 600/319

(57) ABSTRACT

Apparatus for measuring blood glucose concentration comprises a laser source (14) capable of producing a radiation beam from a tunable laser, a collimator (12, 13) for collimating the radiation beam (21) to a small diameter, an optical arrangement (12, 13) for directing the collimated beam (21) through the anterior chamber (23) of an eye (22) in a direction such that it forms a angle of between 75° and 105° to the line of sight (24) of the eye so that the beam does not directly strike the retina (26) and in particular the macular (27). A spectrometer (1-6) is provided to measure the intensity of Raman scattered radiation at different wavelengths. Signal processing means (15) derives the blood glucose concentration from the measured intensities. In one embodiment the laser is tunable between 500 nm. and 700 nm. and the laser beam is collimated to a diameter of between 1 μm and 200 μm.

The invention relates to a method of and apparatus for measuring blood glucose concentration.

Many different approaches for measuring glucose in blood non-invasively have been attempted but none of them has reached the accuracy required for a break through in real application. Measuring the glucose concentrations in the eye for calculating glucose concentration in blood has been proposed as well as different approaches using Raman spectroscopy.

In the first aspect the invention provides a method of measuring blood glucose concentration comprising the steps of;

a) producing a laser beam from a narrow bandwidth laser source, or a tunable laser source, or from a plurality of narrow bandwidth laser sources,

b) collimating the laser beam to a small diameter and passing it through the anterior chamber of an eye in a direction such that the beam does not directly strike the retina nor enter the posterior chamber of the eye (vitreal body),

c) observing Raman scattered light in a direction transverse to the incident laser beam,

d) measuring the intensity of the scattered radiation at different wavelengths, and

e) deriving the blood glucose concentration from the measured intensities.

Measuring the glucose concentration in the eye to enable calculation of the glucose concentration in blood has been proposed using Raman spectroscopy as described in U.S. Pat. Nos. 6,181,957 and 6,424,850. These patents describe a non-invasive method for determining blood level of an analyte of interest, such as glucose, that comprises: generating an excitation laser beam (e.g., at a wavelength of 700 to 900 nanometers); focusing the excitation laser beam into the anterior chamber of an eye of the subject so that aqueous humor in the anterior chamber is illuminated; detecting (preferably confocally detecting) a Raman spectrum from the illuminated aqueous humor; and then determining the blood glucose level (or the level of another analyte of interest) for the subject from the Raman spectrum. Preferably, the detecting step is followed by the step of subtracting a confounding fluorescence spectrum from the Raman spectrum to produce a difference spectrum; and determining the blood level of the analyte of interest for the subject from the difference spectrum, preferably using linear or nonlinear multivariate analysis such as partial least squares analysis. Apparatus for carrying out the foregoing method is also disclosed. The content of these patents is hereby incorporated by reference.

It will be apparent that the geometry of the present invention is different from that shown in the two US patents and has an advantage with respect to safety in that the possibility of damage to the macular is reduced by adopting the geometry of the present invention as the laser beam does not strike the retina. In practice no more than about 15% of the laser power reaches the surface of the macular.

In one embodiment of the invention, a tunable continuous wave laser is used that is tunable in a wavelength range between 500 nm. and 700 nm. The laser beam is preferably collimated to a diameter of between 1 μm and 200 μm and the incident angle of the laser beam is directed at 90°±15° to the line of sight of the eye.

In order to reduce the stray light reaching the detector, the refracted light issuing from the other side of the eye may be absorbed, for example by placing absorbing material over the nose. In certain embodiments the elastically scattered light background illumination florescence and Raman peak of water are also measured and subtracted from the spectra.

In a second aspect the invention provides apparatus for measuring blood glucose concentration comprising a laser source capable of producing a radiation beam form a tunable laser, a collimator for collimating the radiation beam to a small diameter, an optical arrangement for directing the collimated beam through the anterior chamber of an eye in a direction such that it forms an angle of between 75° and 105° to the line of sight of the eye so that the beam does not strike the retina, a spectrometer for measuring the intensity of Raman scattered radiation at different wavelengths, and signal processing means for deriving the blood glucose concentration from the measured intensities.

The above and other features and advantages of the invention will be apparent from the following description, by way of example, of an embodiment of the invention with reference to the accompanying drawings, in which:

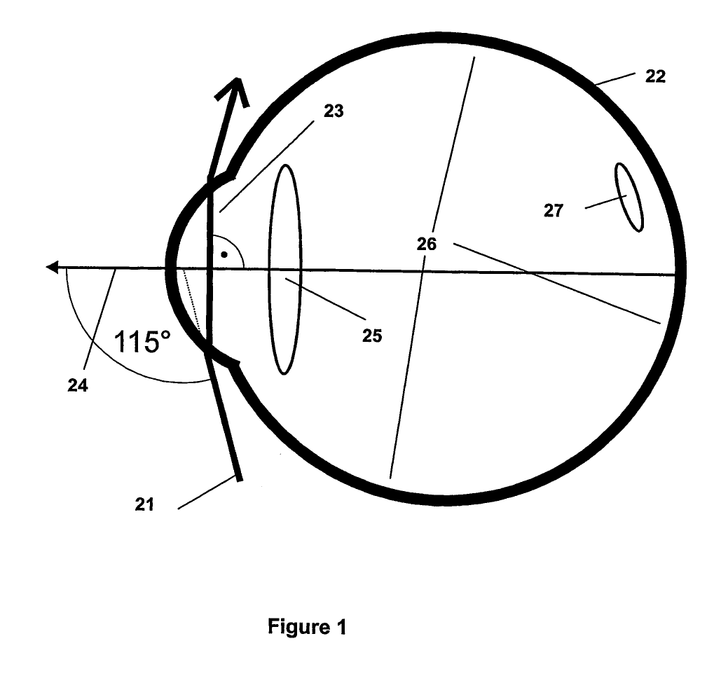

FIG. 1 shows a diagrammatic cross sectional view of an eye and illustrates the geometry of illuminating the anterior chamber of the eye,

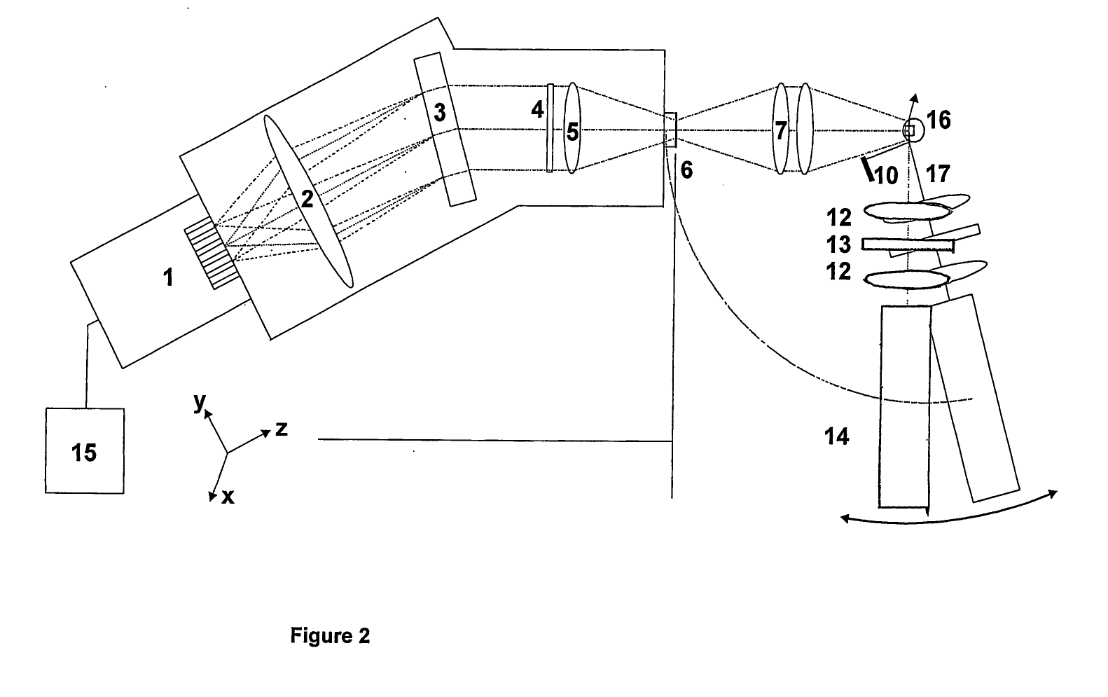

FIG. 2 shows apparatus for measuring blood glucose concentration according to the present invention but having a cylindrical cuvette containing solutions for analyses and mimicking the eye,

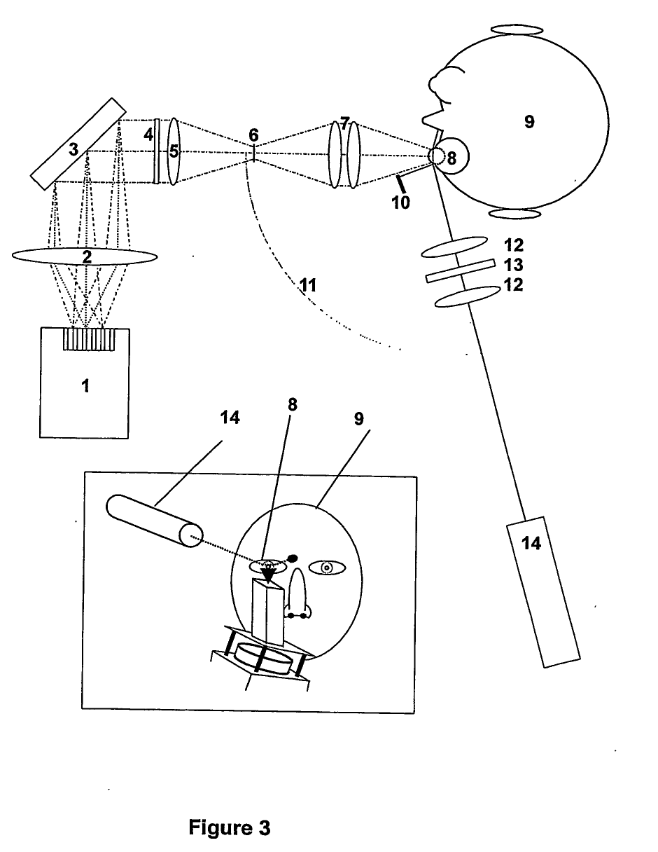

FIG. 3 shows apparatus according to the invention set up to measure the glucose concentration within the eye, and

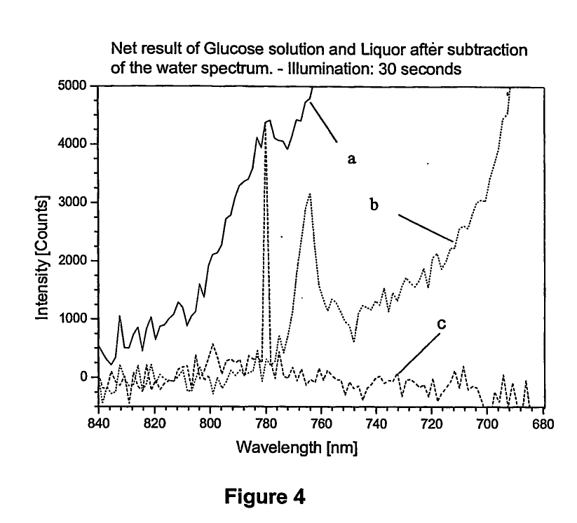

FIG. 4 shows the spectra obtained using the apparatus of FIG. 2.

FIG. 1 shows diagrammatically the path of a beam of radiation 21 from a laser through an eye 22. It will be seen that the beam passes through the anterior chamber 23 of the eye and neither passes directly through the lens 25 nor strike the retina 26 and in particular the macular 27. As can be seen, the laser beam enters the eye at an angle of 115° to the line of sight of the eye.

FIG. 2 shows the apparatus in an experimental set up used to prove the viability of the inventive concept where an eye is replaced by a cylindrical cuvette 16. The apparatus comprises a tunable laser source 14 with focusing optics comprising two lenses 12 and a laser line filter 13. In this schematic the laser and focusing optics are aligned along an axis 17, which coincides with the direction of the beam as it impinges on the cuvette 16. This is clearly not an essential arrangement in that the laser 14 may have optics interposed between it and the focusing lenses to change the direction of the beam. The focusing arrangement is such as to be able to direct the beam along the required line 17. The Raman scattered radiation is focused on a slit 6 by a means of lenses 7 which are arranged along a theoretical line of sight of an eye. The slit 6 comprises the entrance to a spectrometer and radiation is passed through the slit 6 to a lens 5 and a notch filter 4, the lens producing parallel radiation. The term notch filter 4 includes, but is not limited to, a super notch, notch plus, and super notch plus filter

This radiation is passed through a spectrometer grating 3, or alternatively a prism or one or more band pass filters in order to select one or more Raman peaks, and lens 2 to be focused on a detector, for example a CCD camera 1. The output of the detector is fed to a processing arrangement 15, which is capable of storing, and processing the spectra detected and deriving a glucose concentration from them.

A shutter 10 may be provided to minimise reflected light being passed into the spectrometer. In the embodiment shown in FIG. 2 the axis 17 may be rotated with respect to the line of the sight of the eye. The preferred angle lies between 50° and 130°.

In an alternative embodiment instead of a spectrometer, a beam splitter and different optical band pass filters may be used to simplify the set up. The particular form of detection of the Raman scattered light is not crucial to the invention. Thus, in an embodiment of the invention a detector for the non-invasive determination of the blood glucose level by optically measuring the concentration of glucose in the eye comprises a narrow bandwidth laser beam, several lenses and optical filters, a spectrometer, and a detection system

The excitation laser illuminates the anterior chamber of the eye from the direction different from the line of sight. Thus, it does not pass through the lens and is not refracted to the macular. The volume of aqueous humour in the anterior eye chamber being illuminated leads to a Raman spectrum that can be used to determine the glucose concentration. This concentration is related to the glucose concentration in the blood.

Elastically scattered light, background illumination, and fluorescence may be subtracted, as also may be the Raman peak of water. This subtraction can be achieved using the processing arrangement 15 and known techniques.

Particular embodiments of the invention use a tuneable laser that is tuneable over a wavelength band between 500 nm and 700 nm. The laser beam may be collimated to a diameter of 1 μm to 200 μm and passed into the anterior chamber on the eye from the side or from above or below the line of sight. When illumination is from the side, the light that is refracted towards to nose may be suppressed by absorbing material placed over the nose or otherwise between the eye and the nose in order to prevent stray light. Alternatively a photodiode may measure the remaining portion of the illuminating power and this may be used for correction of the detected spectrum.

The light that is directly reflected from the eye surface towards the detector may be blocked by a shutter mechanism or else a small photodiode for determining the lost portion of the incident power may be provided to enable an appropriate correction to be obtained. The Raman scattered light is detected at an angle different from that of the incident beam. The angle may be in the range between 50° and 130°. It should preferably be arranged so that the angle for observing the Raman spectrum is substantially perpendicular to the illumination within the region of interest. A spectrometer or two spatially separated band pass filters or a combination of the two may be used to distinguish to intensities at different wavelengths.

FIG. 4 shows the results of using the apparatus shown in FIG. 2 to determine the spectra of two glucose solutions and a solution of human anterior chamber liquor. Curve a) shows the net spectrum of human anterior chamber liquor after subtraction of the water spectrum, but without subtraction of any stray light or fluorescence. Curve b) shows the next spectrum of glucose solution (for a 2 mg/dl). Curve c) shows the next spectrum of glucose solution (23 mg/dl). The displacement of the glucose peak in curve b originates from a slight displacement of the cuvette relative to the beam. Such a shift of the calibration is easily corrected as a result of the typical Raman fingerprint. FIG. 4 also shows that the glucose concentration in aqueous humour can be measured performing fluorescence subtraction.

FIG. 3 shows essentially the same apparatus as that shown in FIG. 2, but with a head 9 and eye 8 replacing the cuvette 16. The axis 17 corresponds to the laser beam 21 in FIG. 1 and traces the same path through the eye 8 as that shown through the eye 22 in FIG. 1. As can be seen from FIG. 3, the radiation from the laser after passing through the anterior chamber of the eye exits towards the nose and absorbing material may be placed between the eye and nose so that radiation issuing from the eye may be absorbed and not reflected by the nose to the entrance of the spectrometer. This reduces the effect of background radiation and possible saturation of the detector.

1. A method of measuring blood glucose concentration comprising the steps of;
a) producing a laser beam from a narrow bandwidth laser source, or a tunable laser source, or from a plurality of narrow bandwidth laser sources,
b) collimating the laser beam to a small diameter and passing it through the anterior chamber of an eye in a direction such that the beam does not directly strike the retina nor directly enter the posterior chamber of the eye (vitreal body),
c) observing Raman scattered light in a direction transverse to the incident laser beam,
d) measuring the intensity of the scattered radiation at different wavelengths, and
e) deriving the blood glucose concentration from the measured intensities.

2. A method as claimed in claim 1 wherein in step a) a tunable continuous wave laser is used that is tunable in a wavelength range between 500 nm and 700 nm.

3. A method as claimed in claim 1 wherein in step b) the laser beam is collimated to a diameter of between 1 μm and 200 μm.

4. A method as claimed in claim 1 in which in step b) the incident angle of the laser beam is 90°±15° to the line of sight of the eye.

5. A method as claimed in claim 1 including the step of absorbing refracted light issuing from the other side of the eye.

6. A method as claimed in claim 1 in which in step c) the Raman scattered light is observed at an angle of between 50° and 130° to the incident beam.

7. A method as claimed in claim 1 comprising the further step of subtracting fluorescence spectra from the total detected spectra to obtain the Raman spectra.

8. Apparatus for measuring blood glucose concentration comprising a laser source capable of producing a radiation beam from a tunable laser, a collimator for collimating the radiation beam to a small diameter, an optical arrangement for directing the collimated beam through the anterior chamber of an eye in a direction such that it forms an angle of between 75° and 105° to the line of sight of the eye so that the beam does not strike the retina, a spectrometer for measuring the intensity of Raman scattered radiation at different wavelengths, and signal processing means for deriving the blood glucose concentration from the measured intensities.

9. Apparatus as claimed in claim 8 in which the laser is tunable between 500 nm and 700 nm.

10. Apparatus as claimed in claim 8 in which the laser beam is collimated to a diameter of between 1 μm and 200 μm.

11. Apparatus as claimed in claim 8 comprising means for absorbing refracted light issuing from the other side of the eye.

12. Apparatus as claimed in claim 8 in which the spectrometer receives Raman scattered light at an angle of between 50° and 130° to the incident beam.

13. Apparatus as claimed in claim 8 in which the scattered intensity is measured at two or more different wavelengths.
